(19) United States
(12) Patent Application Publication
(10) Pub. No.: US 2009/0281048 A1
(43) Pub. Date: Nov. 12, 2009

(54) Method and System for Inducing Anti-aging in Skin

(76) Inventor: Stephen N. Pitcher, Alpine, UT (US)

Correspondence Address: BATEMAN IP LAW GROUP, P.O. BOX 1319, SALT LAKE CITY, UT, 84110, US

(21) Appl. No.: 12/116,948

(22) Filed: May 7, 2008

(51) Int. Cl. A61K 31/7034; A61K 31/352; G05F 1/10

(52) U.S. Cl. 514/26; 514/456; 315/291

(57) ABSTRACT

A method of treating the skin and accelerating its natural anti-aging and wound-healing processes by using photorejuvenation at a specified total light flux at a specified range of wavelengths from a hand-held light source in combination with the topical ingredients including extracts. Some extracts may be panax ginseng, camellia sinensis, and/or gynostemma pentaphyllum, to effect superior transdermal penetration and intra-cellular delivery, effecting greater bioavailability and bioutilization of some other anti-aging topicals.

### FIELD

This patent application discloses a system and method for treating skin by initiating its wound-healing process and increasing cellular metabolism through usage of a light-emitting device in combination with a trans-dermal and intra-cellular ingredient delivery complex that is combined with select active ingredients to produce a synergistic effect which is greater than that accomplished with either light or dermal cosmetics alone.

### BACKGROUND

Americans spend hundreds of millions of dollars each year in skin-care products to protect and/or revitalize skin. The world market for such beauty-related products is growing and becoming more important. Some products include natural compounds that include beneficial effects. Others have designed and researched new therapies designed to improve skin health, which use light to help repair damaged skin. For example, U.S. patent application Ser. No. 11/660,088 discloses the use of specific pulse wavelengths and photoactive compounds to improve skin conditions, but discloses the use of fairly ineffective wavelengths and photoactive compounds for the stated purpose. Likewise, U.S. Pat. No. 6,676,655 discloses a range of topical ingredients to be used in association with electromagnetic radiation, but has a limited effective period due to a less-than-optimal selection of compounds disclosed. U.S. Pat. No. 7,303,772 discloses some topical compounds for improved skin care, but likewise misses important compounds and amounts, rendering such compounds less effective. U.S. Pat. No. 7,101,385 discloses a method for treating cellulite with phototherapy, but the phototherapy disclosed does not induce angiogenesis and wound-healing response.

U.S. Pat. No. 7,198,634 also teaches the advantages of phototherapy for inducing the nitrous oxide effect of dilating vascular walls, but does so within a limited infrared light source without botanical extracts in combination with topical ingredients, reducing the over-all effectiveness of such a procedure.

Additionally, previous light source devices have either been less effective or ineffective because of incorrect wavelengths emitted and a power output insufficient to sustain beneficial effects of light therapies. Other previous light sources having sufficient power output are large and very expensive, eliminating their potential use in personal care and grooming, requiring instead expensive trips to a dermatologist or other skin care professional. Additionally, the successful use of light alone to promote effective skin rejuvenation requires significant optical energy. This requires a very long treatment time per session, sometimes lasting hours, or uncomfortably high light levels to impart enough optical energy to create the biological conditions necessary for some skin benefits, typically producing modest, but notable cosmetic results.

Thus, previous innovations have relied upon specific pulses of light utilizing a laser or other device, and certain compounds that have limited potential for helping skin improvements. Additionally, each of the current treatments has limited performance, both in the effective time that the treatment lasts, as well as the initial effectiveness.

### SUMMARY

This application discloses a device and associated methods for wound-healing and anti-aging procedures in the skin of an individual. In the presence of specified light wavelengths and topical actives, wound-healing and other beneficial processes conducive to anti-aging may be accelerated through increased action of angiogenesis and vascular endothelial growth. Nitric oxide may also play a large part in angiogenesis, especially through a combination of specific light flux, including particular intensities and wavelengths, and some particular topical actives. Nitric oxide increases the effects of angiogenesis, resulting in significant wound-healing acceleration that results in tighter, firmer, and more wrinkle-free skin, which may also contribute to anti-aging, or maintaining a youthful appearance of the skin.

A light emitter may be used in conjunction with particular topical agents. The light emitter may include many discrete LED devices radiating in specific wavelengths, for example from about 570 nm-1000 nm, or combinations of wavelengths, enclosed in a flexible and compact device of such a size and configuration that it may be easily manipulated by an individual for personal use. When light of the particular wavelengths is absorbed, it may provide accelerated benefits for many biological components in the skin. The absorbed energy may be utilized by nearly every cell in the skin and supporting tissues, providing many beneficial effects.

The light emitter may produce a sensed feeling of warmth that originates from a portion of the absorbed energy converted into heat in the dermis. Biological components in skin produce noticeable anti-aging cosmetic effects when stimulated such as wrinkle reduction and increased skin firmness. In addition, this light therapy produces significant collagen bundle remodeling, which helps to create a more robust and organized ExtraCellular Matrix (ECM) to better support and encompass skin tissue.

Active ingredient transport compounds that are designed to improve trans-dermal penetration and intra-cellular transport and permeation may also be employed with some topical agents. In combination with light therapy, active compounds in a topical agent may be absorbed and utilized more rapidly that without light therapy. Also, a marked and tactile effect has been noted by using the proposed formulations that provide a remarkable long-lasting effect as a result of light-therapy stimulation, lasting up to twelve hours after using the light emitter. Light therapy enhances dermal and cellular transport mechanisms to deliver extended deep penetrating responses when combined with compounds such as panax ginseng, gynostemma pentaphyllum, camellia sinensis, rehmannia glutinosa, rhodiola rosea, and sophora flavescens. Some formulations can produce the proper effect.

Many medium- to large-molecular weight ingredients such as phenolics (e.g., green tea ECCG) and polysaccharides are difficult to pass through the epidermis to the dermis, basal, and sub-dermal regions of the skin. In addition, many active ingredients such as peptides and other compounds can be strongly absorbed in the upper regions of the dermis, reducing supplementation (concentration) to the dermal-epidermal junction, basal layers, and below. The combined use of a light therapy device and topical agents may (1) improve trans-dermal penetration and greatly increase trans-dermal absorption rate, and (2) improve intra-cellular transport and permeation, which increases bioavailability and/or bioutilization of select active ingredients.

One physiological mechanism called gap junction intra-cellular signaling (GJIS) may be enhanced by light stimulation. GJIS is a mechanism by which surrounding cells “communicate” with each other, sending chemical signals that stimulate and promote macro-responses within living tissue. Increasing activity of this mechanism may contribute greatly to overall skin stimulation and utilization of active ingredients. If GJIS is enhanced, then skin tissues may more readily accept and utilize active ingredients, and produce a more homogeneous response. The use of a light therapy device and topical agents as described below purposefully exploits this mechanism in a unique way.

Active ingredients, such as those described herein, can work in synergy with light therapy, and the trans-dermal and intra-cellular delivery complex. When skin tissues are primed with key supportive active ingredients, an enhanced cosmetic response may be achieved. For example, some of these important actives support and extend cellular activity through increased ATP synthesis and oxygen respiration. Other compounds promote collagen tension and remodeling, giving a very noticeable tightening effect after light therapy. This particular response further serves as a tactile effect of the power of light therapy to the consumer. Effective use of peptide complexes in combination with light therapy and the trans-dermal and intra-cellular delivery complex may produce significant anti-aging responses. This is due to enhanced cellular energy present while influencing selected genes to up-regulate or down-regulate certain growth signals. Therefore, wound-healing peptides combined with the light stimulation may provide a superior result than just peptides alone.

These and other aspects of the present invention will become more fully apparent from the following description and appended claims.

### BRIEF DESCRIPTION OF THE DRAWINGS

The following description can be better understood in light of Figures, in which:

FIG. 1 illustrates a light therapy device;

FIG. 2 illustrates the light therapy device of FIG. 1; and

FIG. 3 illustrates a light therapy device in use on a portion of skin on an individual.

Together with the following description, the Figures demonstrate and explain the principles of the skin treatment system and associated apparatus and methods. In the Figures, the thickness and configuration of components may be exaggerated for clarity. The same reference numerals in different Figures represent the same component.

### DETAILED DESCRIPTION

Aspects and features of skin treatment systems including light emitting devices and topical agents are disclosed and described below.

In some embodiments, use of skin treatment systems and associated methods disclosed herein may result in a prolonged increase in capillary circulation and growth of new capillaries, utilizing a method that does not involve the use of skin irritation ingredients, at the same time increasing dermal cellular metabolism to drive this process. This therapy is extremely conducive to tissue regeneration and repair, enhancing the cosmetic effect

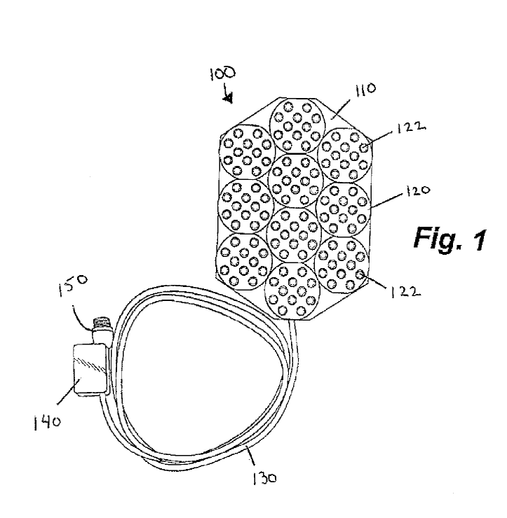

In some embodiments, skin treatment systems and methods may include light therapy device 100 as shown in FIGS. 1-3. Light therapy device 100 may include body 110, a plurality of LED arrays 120; including discrete LEDs 122, cord 130, controller 140, and power supply attach 150. Body 110 maybe designed to be held by an individual in close proximity to treatment site 170 on an individual ISO. Body I 10 may be of any size or dimension consistent with operation and functionality of light therapy device 100 as described below. For example, body 110 may be ergonomically shaped and designed to be easily and comfortably held by an individual on or adjacent to treatment site 170 for a desired period of treatment time. In some embodiments, body 110 may be made of plastic, metal, foam, rubber, glass, or any other material suitable for such uses and functionality. For example, in some embodiments, body 110 may provide an emitting area of light therapy device 100 of between about 6.5 cm² and about 300 cm². In other embodiments, the device emitting area may be about 155 cm² (24 in²). Emitting area may be defined as the dimensional surface area of the light-emitting panel of the light therapy device 100. Because of beam divergence, illuminated area on treatment area 170 will be larger, depending upon the distance light therapy device 100 may be held away from the surface of treatment area 170.

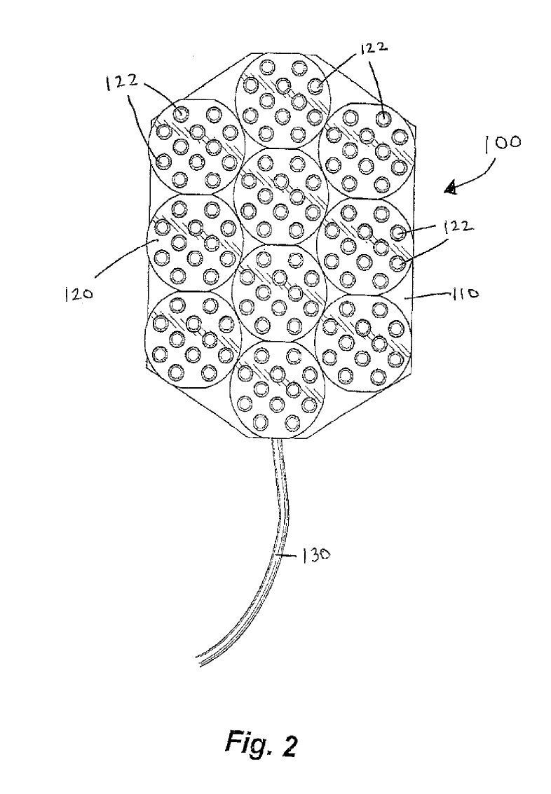

Body 110 may be configured to support, position, and/or hold LED arrays 120. LED arrays 120 may include a plurality of discrete LEDs 122. Body may hold several LED arrays 120. For example, in FIG. 2, light therapy device 100 includes ten LED arrays 120, each LED array containing 12 discrete LEDs 122. In some embodiments, body 110 may include between about two and 50 LED arrays 120, or as many or as few as allows functioning of light therapy device.

LED arrays 120 may be arranged in any manner on body 110 to allow for sufficient light energy to be directed to a treatment area, as described previously and below. Each LED array 120 may include at least two LEDs 122 and as many as desired by one of ordinary skill. LEDs 122 may be any LED or similar device that emits narrowband, multichromatic electromagnetic radiation in the desired spectrum ranges, described in further detail below, such as standard LEDs 122, a laser, a fluorescent light source, an organic light-emitting diode, a light-emitting polymer, a xenon arc lamp, a metal halide lamp, a filamentous light source, an intense pulsed light source, a sulfur lamp, or other similar sources with an output in the desired ranges. LEDs 122 may be functionally attached to each LED array 120 as desired by one of ordinary skill. For example, LEDs 122 may be in series or in parallel in each LED array 120. Similarly, each LED array 120 may be functionally attached to light therapy device 100 in series or in parallel with each other, depending on the circuit design and preference of one of ordinary skill.

LEDs 122 may have wavelengths between about 570-1000 nm. In some embodiments, LEDs 122 may have a range of between about 830-880 nm. LEDs 122 may be selected such that some of LEDs 122 have slightly different wavelengths than other of LEDs 122, depending on the desired wavelengths to be applied. Similarly, LEDs 122 with particular wavelengths may be selected based on their effectiveness with a particular compound or ingredient in a selected topical agent 160. For example, light therapy device 100 may use LEDs 122 each having a peak wavelength of 850 nm because of preferred quantum efficiency. In some embodiments, the spectral bandwidth of LEDs 122 may have a FWHM (full width half maximum) of about 10 to 60 nm.

In some embodiments, light therapy device 100 may output emitted radiometric optical power as measured on the surface of treatment area 170 of between about 16.7 mW/cm² and about 120 mW/cm². In some embodiments, the emitted radiometric optical power may be about 40 mW/cm² and about 80 mW/cm². In other embodiments, the emitted radiometric optical power of light therapy device may be about 60 to 80 mW/cm². Power below about 16.7 mW/cm² may be less desirable because light reactive agents in topical agent 160 may be under utilized as some agents tend to degrade with time, limiting the amount of beneficial biological activity available with light emitted from light therapy device 100 and topical agent 160. Power above about 120 mW/cm² may also be undesirable as such a power level may produce uncomfortable tissue heating in treatment area 170 following application of light therapy device 100.

Controller 140 may be a current controller that regulates the current supplied to LED arrays 120 and LEDs 122. Power supply attach 150 may be designed to receive power from any conventional power supply, as desired by one of ordinary skill. For example, power supply attach may be connected to a conventional AC adapter, which provides DC power from a standard AC source, such as a 120V residential power source. Controller 140 may be a solid state device, or may be any device that functions to control the supply of power to LED arrays 122 consistent with the desired function of light therapy device 110 as described further below.

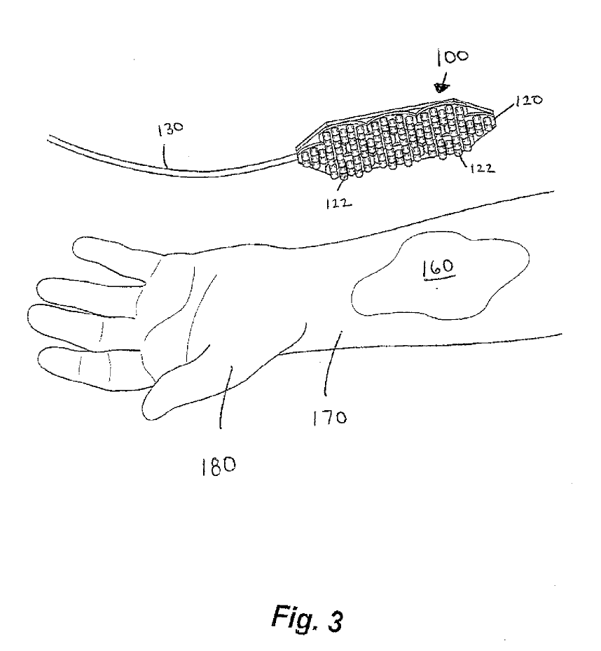

FIG. 3 illustrates an embodiment of usage of light therapy device 100 on a treatment area 170 of an individual 180 (ere, treatment area 170 is located on the forearm of the individual 180 receiving the therapy). Topical agent 160 may be applied to treatment area 170 prior to applying light therapy device 100. Topical agent 160 may be applied on and massaged into the skin of treatment area 170 until absorbed.

As shown in FIGS. 3 and 4, the skin treatment system may be used to provide treatment to an individual by applying topical agent 160 to treatment area 170. A user or skin-care professional may select topical agent 160 for use with light therapy device 100. Topical agent 160 may be selected based on ingredients, active agents, and effectiveness in conjunction with light therapy device 100, beneficial compounds, etc. Specific examples of desirable topical agents for use with light therapy device 100 are further discussed below at length. Topical agent 160 may be applied to treatment area 170.

Light therapy device 100 may be placed above treatment area 170 for a period of time, for example between about 5 and 30 minutes, which may be selected depending on the power output of light therapy device 100 and the particular topical agent 160 selected. Light therapy device 100 may be removed from treatment area 170. In some embodiments, treatment area 170 may be cleaned and treated further with topical agent 160, another different topical agent, and/or light therapy device 100

In some embodiments, the total energy absorbed by treatment area may determine the effectiveness of the treatment. For example, in some embodiments, a total energy flux of between about 10-140 J/cm² during a single treatment session may be effective to improve skin health and promote regeneration and healing. However, a low energy output of a light source requires that the light source be present for a significant period of time to accrue the desired total energy flux, leading to user discomfort. Similarly, some compounds in topical agent 160 may have a limited time of bio-effectiveness and/or bio-availability, as the compounds react and are utilized. Thus, a sufficiently high total energy output in light therapy device should be sufficient to allow maximum effectiveness of the combination of some embodiments of topical agent 160 and light therapy device 100, reducing the time needed in a single treatment session and maximizing the bio-effectiveness of topical agent 160.

During and after treatment, for up to twelve hours later, individual 180 may experience a significant rejuvenating and tactile effect in the skin such as tingling, extended warmth, pulsations, etc. in treatment area 170. The treatment process may be continued 3 to 4 times per week for 4 to 8 weeks. Repeating treatments resume every 1 to three months after initial treatment is completed and can continue indefinitely until the desired results are attained. Additionally, light energy from light therapy device 100 may fortify the skin of younger people in their twenties and thirties against the cumulative effects of photo-aging. This light therapy can produce compounding benefits for up to two months after completing a round of treatment with the light emitter. The emitter is ideally used up to three or four times per week for the first month of treatment. At normal optical power, the treatment interval is repeated every other month to two months. If reduced optical power is desired or necessary, then the light therapy should be repeated every other month.

A major advantage of the use of light having a wavelength between about 570-1000 nm is the ability for deep tissue penetration—up to 25 mm or more. Most topically applied compounds and agents do not penetrate more than 1 mm to 4 mm. In some embodiments, skin treatment systems with light therapy device 100 and topical agent 160 may create a deep and long-lasting cosmetic effect when combined to allow for deep penetration of bio-effective agents in topical agent 160 due to the effects of topical agent 160 and light therapy device 100, allowing for deeper penetration of topical agent 160 than without use of light therapy device 100, allowing for a trans-dermal and intra-cellular delivery complex. This is partially due to increased energy delivered to cellular tissue from light therapy, and the resulting vasodilation effect that the combined topical and light therapy produce.

Some compounds that may be included in topical agent 160 for specific benefits along with light therapy device may include gynostemma pentaphyllum and panax ginseng. Ginsenosides from panax ginseng may be derived from common ginseng (e.g., panax ginseng or Korean ginseng), and have proven to be very effective in activating potassium channels in smooth muscle cells through nitric oxide pathways, active participants in tissue regeneration, and active in modulating angiogenesis. In particular, certain components of ginsenosides may be particularly effective in the angiogenic process. For example, Re is active in the nitric oxide pathway of angiogenesis; Rg1 is active in human umbilical vein endothelial cell (HUVEC) proliferation; Rg3 induces smooth muscle relaxation and inhibits phenylephrine-induced vascular contraction; and Rd attenuates neuroinflammation of dopaminergic cells; each of which has an effect in the wound-healing process. In addition, other ginsenoside components may play parts in the Ca²⁺-activated K⁺ channels in endothelial cells, which may also play a part in the wound-healing process. In some embodiments, topical agent 160 having extracts of panax ginseng containing from about 5% to 35% ginsenosides.

In some embodiments, a topical active that may be included in topical agent 160 is the extract of camellia sinensis, also proven to have angiogenic effects in part from the phenolic compounds. This, combined in specific proportions with the extract of gynostemma pentaphyllum, has proven remarkable benefits that have been seen above and beyond those observed by any of the single ingredients alone, and in combination with light therapy device 100, provide synergistic effects not predicted or provided for by previous technologies. In some embodiments, topical agent 160 may include extracts from camellia sinensis containing from about 5% to 45% epigallocatechin gallate (EGCG) by volume. Other particularly effective compounds for use in topical agent 160 may include one or more of extracts of gynostemma pentaphyllum containing from about 5% to 45% gypenosides by volume, rehmannia glutinosa containing from about 1% to 2% catapol by volume, rhodiola rosea containing from about 1% to 5% rosavins by volume, and/or about 0.25% to 2% salidroside by volume, and sophora flavescens containing from about 5% to 16% alkaloids by volume. Similarly, topical agent 160 may include any one of or combination of each of the extracts described above.

These specified topical actives may be combined with ingredients from another set of compounds that are more widely known for repairing aged and damaged skin, such as vitamins, peptides, fultlerenes, and other active ingredients in topical agent 160. For example, topical agent 160 may include one or more extracts of compounds selected from the group consisting of peptides, retinoids, yeast extracts, anti-oxidants including fullerenes, anti-oxidant fruit extracts such as oligamerized lychee and green tea extracts, Mangosteen extract (garcinia inangostana), Acai extract (euterpe oleracea), Wolfberry (lycium barbarur) extract, skin moisturizers and humectants, fatty acids and fatty acid oils, chlorophyll-containing compounds, carotenoid-containing compounds, phyocobilin compounds, indocyanine green, methylene blue, rose Bengal, vitamin C, vitamin E, vitamin D, vitamin A, vitamin K, vitamin F, Retin A (Tretinoin), Adapalene, retinal, hydroquinone, kojic acid, a growth factor, Echinacea, an antibiotic, an antifungal, an antiviral, a bleaching agent, an alpha hydroxy acid, a beta hydroxy acid, salicylic acid, antioxidant triad compound, a seaweed derivative, a salt water derivative, algae and other derived algae extracts, phytoanthocyanin, a phytonutrient, plankton, a botanical product, a herbaceous product, a hormone, an enzyme, a mineral, a cofactor, insulin, minoxidil, lycopene, a natural or synthetic melanin, a metlloproteinase inhibitor, proline, hydroxyproline, an anesthetic, chlorophyll, bacteriochlorophyll, copper chlorophyllin, chloroplasts, carotenoids, phycobilin, rhodopsin, anthocyanin, inhibitors of ornithine decarboxylase, inhibitors of vascular endothelial growth hormone, inhibitors of phospholipase A2, inhibitors of S-adenosylmethionine, licorice, licochalone A, genestein, soy isoflavones, phytoestrogens, derivative, analogs, homologs, and subcomponents thereof and derivatives, subcomponents, immunological complexes and antibodies of the target skin, and synthetic and natural analogs therof, and combinations thereof. I combination with the other compounds discussed above, these secondary ingredients may be utilized more effectively than they would be without the presence of the primary ingredients, and exhibit greater bioavailability, which produces better cosmetic results for the user.

Thus, the combined use of light therapy device 100 and topical agent 160 may greatly reduce the required treatment duration and device cost over traditional light treatment devices alone. The combination of light therapy device 100 and topical agent 160 together may result in increased utilization of active skin care ingredients in topical agent 160 by up to two times or more through greater tissue absorption, bioavailability, and/or bio-utilization than without light therapy device 100.

For example, cellular mitochondria may receive additional energy through light stimulation of the cytochrome-C oxidase enzyme. This energy may increase ATP synthesis of cells deep within the tissues for an extended period of time. Also, nitrous oxide may be produced, which relaxes capillary blood vessels, increasing microcirculation, enhancing cellular respiration, and encouraging fluid drainage in the dermis. Fibroblast cells are particularly stimulated from proper light therapy, creating new collagen and elastin. This action is important in maximizing benefits when combined with targeted active ingredients in the formulations. Certain positive growth factors may also be expressed, and several key inflammatory and other destructive compounds may be reduced in the presence of light. The dermal complex also experiences a reduction in MMP proteolytic activity, preserving newly constructed supportive tissues when treated with light therapy device 100 and topical agent 160.

Having described the preferred aspects, it is understood that the invention defined by the appended claims is not to be limited by particular details set forth in the above description, as many apparent variations thereof are possible without departing from the spirit or scope thereof.

What is claimed is:

1. A method, comprising:
applying a topical agent to a treatment area of an individual; and
exposing the treatment area to a light source with an energy output of between about 16 mW/cm² to 120 mW/cm² within the range of wavelengths from between about 570 to 1000 nm, wherein the light source is configured to be hand-held.

2. The method of claim 1, wherein said light source is a source of narrowband, multichromatic electromagnetic radiation selected from a light-emitting diode, a laser, a fluorescent light source, an organic light-emitting diode, a light-emitting polymer, a xenon arc lamp, a metal halide lamp, a filamentous light source, an intense pulsed light source, a sulfur lamp, and combinations thereof.

3. The method of claim 1, wherein the light source is configured to function cooperatively with the topical agent to increase skin health in the treatment area.

4. The method of claim 1, wherein the topical agent comprises extracts of panax ginseng containing from about 5% to 35% ginsenosides.

5. The method of claim 1, wherein the application of the topical agent is performed prior to the exposing the individual to the light source, wherein the topical agent comprises extracts of camellia sinensis containing from about 5% to 45% epigallocatechin gallate.

6. The method of claim 1 further comprising the usage of a topical agent to the skin prior to utilization of said light source, wherein the topical agent further comprises the extracts of gynosremma pentaphyllum containing from about 5% to 45% gypenosides.

7. The method of claim 1 further comprising the usage of a topical agent to the skin prior to utilization of said light source, wherein the topical agent further comprises the extracts of rehmannia glutinosa containing from about 1% to 2% catapol.

8. The method of claim 1 further comprising the usage of a topical agent to the skin prior to utilization of said light source, wherein the topical agent further comprises the extracts of rhodiola rosea containing from about 1% to 5% rosavins and about 0.25% to 2% salidroside.

9. The method of claim 1 further comprising the usage of a topical agent to the skin prior to utilization of said light source, wherein the topical agent further comprises the extracts of sophora flavescens containing from about 5% to 16% alkaloids.

10. The method of claim 1 further comprising the usage of a topical agent to the skin prior to utilization of said light source, wherein the topical agent further comprises one or more extracts of compounds selected from the group consisting of peptides, retinoids, yeast extracts, aunt-oxidants including fullerenes, anti-oxidant fruit extracts such as oligarnerized lychee and green tea extracts, Mangosteen extract (garcinia mangostana), Acai extract (euterpe oleracea), Wolfberry (lycium barbarum) extract, skin moisturizers and humectants, fatty acids and fatty acid oils, chlorophyll-containing compounds, carotenoid-containing compounds, phyocobilin compounds, indocyanine green, methylene blue, rose Bengal, vitamin C, vitamin F, vitamin D, vitamin A, vitamin K, vitamin F, Retin A (Tretinoin), Adapalene, retinal, hydroquinone, kojic acid, a growth factor, Echinacea, an antibiotic, an antifungal, an antiviral, a bleaching agent an alpha hydroxy acid, a beta hydroxy acid, salicylic acid, antioxidant triad compound, a seaweed derivative, a salt water derivative, algae and other derived algae extracts, phytoanthocyanin, a phytonutrient, plankton, a botanical product, a herbaceous product, a hormone, an enzyme, a mineral, a cofactor, insulin, minoxidil, lycopene, a natural or synthetic melanin, a metlloproteinase inhibitor, proline, hydroxyproline, an anesthetic, chlorophyll, bacteriochlorophyll, copper chlorophyllin, chloroplasts, carotenoids, phycobilin, rhodopsin, anthocyanin, inhibitors of ornithine decarboxylase, inhibitors of vascular endothelial growth hormone, inhibitors of phospholipase A2, inhibitors of S-adenosylmethionine, licorice, licochalone A, genestein, soy isoflavones, phytoestrogens, derivative, analogs, homologs, and subcomponents thereof and derivatives, subcomponents, immunological complexes and antibodies of the target skin, and synthetic and natural analogs therof, and combinations thereof.

11. The method of claim 1, wherein the exposing results in a flux of between about 10-140 J/cm² during a treatment session.

12. The method of claim 1, wherein the exposing is performed for between about 5 and 30 minutes.

13. The method of claim 1, wherein the light source includes at least one array of LEDs controlled by a constant current device.

14. The method of claim 13, wherein the at least one array of LEDs includes between about 2 and 100 LEDs.

15. The method of claim 14, wherein the at least one array of LEDs is at least two arrays, each array having between about 2 and 100 LEDs.

16. The method of claim 13, wherein at least a portion the array of LEDs are configured to emit wavelengths different from the wavelength of at least another portion of the array of LEDs.

17. The method of claim 1, wherein the treatment area is on the face of the individual.

18. A light emitting device, comprising
a body configured to be held and manipulated by an individual;
a plurality of LEDs, wherein the plurality of LEDs are configured to output a combined energy output of between about 16 mW/cm² to about 120 mW/cm² within the range of wavelengths from between about 570 to 1000 nm; and
a controller configured to control light output of the plurality of LEDs.

19. The light emitting device of claim 18, wherein the controller is a current controller.

20. The light emitting device of claim 18, wherein each of the plurality of LEDs has a peak wavelength of about 850 nm and a full width half maximum of about 10 to 60 nm.

21. The light emitting device of claim 18, wherein the combined energy output is about 60 to 80 mW/cm².
